(19) United States
(12) Patent Application Publication
(10) Pub. No.: US 2003/0158475 A1
(43) Pub. Date: Aug. 21, 2003

(54) Intravaginal radiofrequency imaging device

(76) Inventors: Vicki Young Johnson, Hoover, AL (US); Edward G. Walsh, Irondale, AL (US); Bradley R. Newcomer, Birmingham, AL (US); Mary Grace Umlauf, Birmingham, AL (US)

Correspondence Address: Benjamin Aaron Adler, ADLER & ASSOCIATES, 8011 Candle Lane, Houston, TX, 77071, US

(21) Appl. No.: 10/373,226

(22) Filed: Feb. 24, 2003

Related U.S. Application Data
(63) Continuation-in-part of application No. 09/822,720, filed on Mar. 30, 2001, now Pat. No. 6,526,306.
(60) Provisional application No. 60/193,229, filed on Mar. 30, 2000.

(51) Int. Cl. A61B 5/05

(52) U.S. Cl. 600/410

(57) ABSTRACT

The present invention provides a vaginal imaging device for quantifying morphological and biochemical changes in the pelvic floor muscles as well as monitoring muscular function. Specifically, the present invention provides a vaginal imaging probe comprising a single or dual tuned resonator for both nuclear magnetic resonance imaging and spectroscopy of the pelvic floor musculature.

### CROSS-REFERENCE TO RELATED APPLICATIONS

This is a continuation-in-part of non-provisional patent application U.S. Ser. No. 09/822,720, filed Mar. 30, 2001, which claims benefit of provisional patent application U.S. Serial No. 60/193,229, filed Mar. 30, 2000, now abandoned.

### BACKGROUND OF THE INVENTION

1. Field of the Invention

The present invention relates generally to the fields of medical devices and medical diagnostics and treatment. More specifically, the present invention relates to an intravaginal radiofrequency imaging device for intravaginal monitoring to assess the function, morphology, and exercise-induced metabolic and biochemical changes in the pelvic floor muscles surrounding the vaginal vault.

1. Description of the Related Art

Magnetic resonance imaging can be used for imaging of physiologic function, in addition to anatomical imaging. One such area is in the imaging of muscular function, both cardiac and skeletal muscle. A method for quantifying the contractile function of the heart is known as radiofrequency (RF) tagging. In this method, image data readout is preceded by a composite RF excitation that produces a series of dark parallel lines in the image. These lines result from the selective saturation of tissue within the field of view (FOV). In cardiac imaging, this excitation would be delivered on the R-wave trigger, i.e. at end diastole. Since material points in the tissue have been saturated, the lines are seen during image playback to move with the tissue as the heart contracts. Two such excitations can be used on the R-wave trigger to produce a grid of lines.

An important feature of this method is that such images can be analyzed using automated techniques to track the tag line motion, and thus produce maps of strain and shear, as well as strain and shear rates. It is also possible to produce strain and shear maps illustrating the function of skeletal muscle when a triggering signal representing a reproducible stimulus can be produced. Contractile force or pressure is such a reproducible stimulus.

The pelvic floor muscles provide support for the bladder, bladder neck and urethra. Urinary leakage occurs due to hypermobility of the urethra subsequent to a laxity of these muscles. This results in inadequate urethral compression during increases in intra-abdominal pressure such as with coughing, rising from a seated position, or exercising. Exercise to recondition the muscles of the pelvic floor is not a new concept. Specificity of training is paramount to achieve optimal functioning of the muscle for its intended use (1-2). Muscles of the levator ani, collectively called the pelvic floor musculature (PFM), are a heterogeneous mixture of 70% Type I slow twitch fibers and 30% Type II fast twitch fibers (3-5). Type II muscle fibers are further delineated into Type Ia and Type IIb fibers. Type IIa fibers have a preponderance of glycolytic enzymes in their mitochondria, are larger in diameter and fatigue very quickly. In contrast, Type IIb fibers have fewer glycolytic enzymes in their mitochondria, are smaller in diameter, and are more resistant to fatigue.

Kegel (6) introduced pelvic floor musculature exercises four decades ago with reported 69-93% success rates in treating females with stress urinary incontinence (SUI) (8-10). Studies that have examined muscle response to training have targeted Type II muscle fibers in strength-training regimens to recondition the pelvic floor musculature (11-13). However, these investigators merely hypothesized the mechanism for improvement as being exercise-induced hypertrophy because studies to describe the pelvic floor musculature in regard to muscle fiber type and mechanism of action have been limited to in vivo biopsy at the time of surgery or cadaver dissection (4).

Although there have been many advancements in the treatment of urinary incontinence using pelvic floor muscle exercises within a behavioral framework, investigators have been unable to describe the precise mechanisms of improvement. There are many potential and competing theories for the mechanisms of action responsible for recovery of continence. Some have hypothesized that increasing muscle strength allows the patient better sphincter control while others have suggested that with exercise the muscle size increases to provide additional occlusive bulk around the urethral sphincter.

There is little agreement on the correct technique for performing pelvic floor muscle exercise (14). Additionally, few studies have been undertaken to determine contraction intensity level of exercise to ensure success in pelvic floor muscle exercise therapy (13). Furthermore, no in vivo studies have shown the dynamic biochemical and metabolic changes that occur during or resulting from pelvic floor muscle exercise.

Most exercise protocols to improve function of the pelvic floor muscle have targeted strength enhancement and have been successful in decreasing leakage episodes. Descriptions of specific muscular responses resulting in functional changes of the pelvic floor muscle are inconsistent between studies. Factors attributed to functional changes include increased vaginal pressures, lengthening of the functional area of the urethra, and initial neural adaptation.

One study used graded pelvic muscle exercises to strengthen the pelvic floor muscle and enhance endurance of muscle contractions in 65 women aged 35-75 years (13). This 16-week exercise protocol required exercises three times per week with measurements taken every 4 weeks. Gradation of the exercises involved maximal contraction effort that increased in number of contractions over the protocol period. Endurance exercises were maximum effort with emphasis on sustaining the contraction for 10 seconds. The investigators hypothesized that sustained pressure would benefit the Type I muscle fibers, while the repeated maximum contraction effort would benefit the Type II muscle fibers.

Decreases in grams of urine loss were statistically significant (t=−4.7, p<0.0001). Episodes of leakage in24 hours decreased from 2.6 to 1.0. No statistically significant correlation between urine loss and maximum pressure or between urine loss and sustained pressure was found. The investigators suggested that this finding might indicate that the mechanisms of pelvic floor muscle exercise affecting SUI are not explained by pressure changes alone. In fact findings in a recent study (N=32) indicated that submaximal exercise not only increased endurance and resulted in decreases in quantity of urine leakage, but also was significantly more effective (t=1.75; p=0.045) for increases in strength of contraction effort than using a near-maximal exercise protocol (15).

Technology is needed to enable investigators to describe the mechanisms responsible for improvement in continence. The ability to analyze the regional mechanical and metabolic changes that occur in the pelvic floor muscle as a result of exercise might facilitate determining which exercise protocols are more effective and at what intensity the greatest improvement occurs. Strain and shear maps can reveal the presence of asymmetric function, or a subtle muscle injury such as internal perineal tear from birth injury. Investigation of the phosphorus metabolites and the pH would provide a chemical “snap-shot” of the cellular metabolism which can reveal abnormalities such as reduced perfusion. However, current technology requires tissue biopsy to conduct this type of analysis. Nuclear MRI and spectroscopy, using state-of-the-art techniques, to describe changes in the pelvic floor muscle structurally during exercise and to conduct biochemical and metabolic analyses as subjects exercise and improve over time, might give researchers insight into the mechanisms responsible for change and improvement without the need for invasive biopsy.

MRI has been used to visualize pelvic floor muscle contraction in normal females (N=6). Findings showed that PFM contractions using MRI could be identified and that anatomical displacement of the bladder could be demonstrated. This study used coronal and sagittal planes for imaging (16). However, no study has reported use of an insertable vaginal device with incorporation of force transduction and phosphorus spectroscopy to allow investigators the ability to conduct force of contraction measurements and biochemical analyses without the need for tissue biopsy.

The prior art is deficient in the lack of a non-invasive device/means of intravaginal monitoring. Specifically, the prior art is deficient in the lack of an intravaginal imaging device for monitoring and conducting biochemical analysis such that the imaging device combines a NMR resonator and force transduction mechanism in a single device. The present invention fulfills this long-standing need and desire in the art.

### SUMMARY OF THE INVENTION

In one embodiment of the present invention, there is provided a vaginal imaging device comprising a single or dual tuned resonator comprising a transmit/receive element for nuclear magnetic resonance imaging and spectroscopy; a tuning/matching circuit remote from the resonator; and a force transduction mechanism for monitoring a subject's contraction effort and to trigger said resonator to produce vaginal imaging and spectroscopy data.

In another embodiment of the present invention, there is provided a vaginal imaging device comprising a single tuned or dual tuned resonator for nuclear magnetic resonance imaging or nuclear magnetic resonance and spectroscopy comprising a Helmholz array; a tuning/matching network remote from said resonator; and an optical force transducer that generates a gating signal from an optical signal proportional to developed force, said gating signal triggering said resonator to produce vaginal imaging and spectroscopy data.

In other embodiments of the present invention, there are provided methods of imaging and assessing biochemical states in pelvic floor musculature in situations such as before and after exercise, before and after surgical repair and before and after pharmaceutical therapy in individual suffering from abnormalities in pelvic floor musculature using the vaginal imaging devices described herein.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 depicts the layout of the components for the housings of a probe containing a single turn solenoid antenna and a pneumatic pressure transducer.

FIG. 2 depicts the layout and geometry of the disposable portion of the probe.

FIG. 3A depicts a radiofrequency tuning/matching circuit detailing the antenna element with the phosphorus reference.

FIG. 3B depicts the printed circuit board layout of the tuning/matching circuit.

FIG. 4 depicts the single turn solenoid antenna element with phosphorus reference.

FIG. 5 depicts the radiofrequency tuning/matching circuit incorporating active decoupling for use as a receive only resonator.

FIG. 6 depicts the interconnections of components for use of the vaginal imaging probe using a pneumatic pressure transducer.

FIG. 7A depicts a Helmholz array probe with fiber optic force transduction mechanism and remote tuning arrangement.

FIG. 7B depicts an enlargement of the panel cover of the remote tuning box.

FIG. 7C depicts the remote tuning box layout.

FIG. 8 depicts the circuit diagram for the optical force transduction mechanism.

### DETAILED DESCRIPTION OF THE INVENTION

In one embodiment of the present invention, there is provided a vaginal imaging device comprising a single or dual tuned resonator comprising a transmit/receive element for nuclear magnetic resonance imaging and spectroscopy; a tuning/matching circuit remote from the resonator; and a force transduction mechanism for monitoring a subject's contraction effort and to trigger said resonator to produce vaginal imaging and spectroscopy data. In one aspect of this embodiment the transmit/receive element is a single turn solenoid oriented to permit non-gradient localized spectroscopy. In another aspect of this embodiment the transmit/receive element is a Helmholz array located around the long axis of the device. In this aspect the array provides a different spatial sensitivity profile than that provided by a single turn solenoid.

In all aspects of this embodiment the tuning/matching circuit is electrically connected to the resonator via a coaxial transmission line having an electrical length of λ/2 wavelength or an integer multiple thereof. The single tuned resonator is for nuclear magnetic resonance imaging using the ¹H isotope and the dual tuned resonator further performs performing nuclear magnetic resonance spectroscopy of a second isotope. Representative examples of the second isotope are ³¹P, ¹³C, ²³Na, ³⁹K, and ⁴³Ca. Additionally, in all aspects a vial containing a 300 mM inorganic phosphate reference solution is located at the center of the transmit/receive element in the resonator to allow chemical shift referencing for the signals obtained.

Further to these aspects the means to trigger the resonator can be provided by an optical force transducer, a piezoelectric force transducer, resistive force transducer or pneumatic pressure transducer. Such a vaginal imaging device is useful for radiofrequency tagged magnetic resonance imaging, phase velocity mapping and diffusion weighted imaging. In a dual tuned resonator non-gradient localized phosphorus spectroscopy may be performed.

Also in all aspects of this embodiment the force transduction mechanism is used to monitor contraction effort of the subject for the purpose of synchronizing scanner image or spectroscopy data acquisition with the contraction effort of the subject. The scanner image may be synchronized with the scanner body volume. The scanner image or spectroscopy data acquisition may be synchronized with its own antenna element in a transmit/receive mode or with its own antenna element in a receive only mode using active or passive decoupling such that decoupling prevents local retransmission of the radiofrequency signal and excessive tissue heating.

In another embodiment of the present invention, there is provided a vaginal imaging device comprising a single tuned or dual tuned resonator for nuclear magnetic resonance imaging or nuclear magnetic resonance and spectroscopy comprising a Helmholz array; a tuning/matching network remote from said resonator; and an optical force transducer that generates a gating signal from an optical signal proportional to developed force, said gating signal triggering said resonator to produce vaginal imaging and spectroscopy data. In this embodiment the remote tuning/matching circuit is, the isotopes used in the nuclear magnetic resonance imaging/spectroscopy and the phosphorus standard are as described supra. An example of the nuclear magnetic resonance imaging is radiofrequency tagged magnetic resonance imaging.

In yet another embodiment of the present invention, there is provided a method for imaging pelvic floor musculature in a subject, comprising the step of applying the vaginal imaging devices disclosed herein to the subject, thereby producing an image of the pelvic floor musculature.

In yet another embodiment of the present invention, there is provided a method of obtaining spectroscopic information on the biochemical state of the pelvic floor musculature by applying the vaginal imaging devices disclosed herein to a subject to produce magnetic resonance spectroscopic information that provides assessment of muscular biochemical activity.

In yet another embodiment of the present invention, there is provided a method of assessing biochemical changes under exercise conditions in pelvic floor musculature by applying the vaginal imaging devices disclosed herein to a subject to acquire magnetic resonance spectroscopic data before, during and after the exercise conditions.

In yet another embodiment of the present invention, there is provided a method of evaluating efficacy of a surgical repair in pelvic floor musculature in an individual by applying the vaginal imaging devices disclosed herein to such individual before and after the surgical repair.

In yet another embodiment of the present invention, there is provided a method for evaluating efficacy of an exercise therapy in an individual by applying the vaginal imaging devices disclosed herein to such individual before and after the exercise therapy.

In yet another embodiment of the present invention, there is provided a method for evaluating efficacy of a pharmaceutical therapy in an individual suffering from abnormalities in pelvic floor musculature by applying the vaginal imaging devices disclosed herein to such individual before and after the pharmaceutical therapy.

The following terms shall be interpreted according to the definitions set forth below. Terms not defined infra shall be interpreted according to the ordinary and standard usage in the art.

As used herein, “pelvic floor musculature (PFM)” shall refer to the levator ani muscles, specifically the coccygeous, pubococcygeous, and iliococcygeous through which the sphincter vaginae and compressor urethrae pass.

As used herein, “piezoelectric force transducer” shall refer to a device that converts applied force into a force-proportional electrical signal.

As used herein, “resistive force transducer” shall refer to a device in which resistance varies with applied force, either through incorporation of a force dependent resistance in a bridge circuit, i.e., Wheatstone bridge, or through use of a material whose intrinsic resistance varies with pressure.

As used herein, “pneumatic pressure transducer” shall refer to a device that converts air pressure into a pressure proportional voltage.

As used herein, “contractile force perpendicular” shall refer to force applied perpendicular to the long axis of the vaginal imaging probe.

As used herein, “single-turn solenoid” shall refer to the radiating and receiving element of the vaginal imaging probe.

As used herein, “Helmholz array” or “Helmholz configuration shall refer to a configuration with two elements, normally designed to produce a homogeneous magnetic field between the elements, but in this application taking advantage of the relatively uniform response produced around the outside of the array. This configuration provides for a more uniform transmit-receive sensitivity around the circumference of the probe.

As used herein, “non-gradient, localized spectroscopy” shall refer to spectroscopy in which data is acquired from the entire sensitive volume of the vaginal imaging probe.

As used herein, “radiofrequency (RF) tagged image” shall refer to images in which a grid of dark tag lines is produced in a series of images to permit assessment of regional tissue motion.

As used herein, “phase velocity mapping” shall refer to an MR imaging technique in which motion sensitizing gradient pulses are used to produce images in which pixel intensities correspond to velocity of blood or tissue during the imaging acquisition.

As used herein, “diffusion weighted imaging” shall refer to an MR imaging technique in which gradient pulses are used to sensitize an image acquisition to random diffusion. This method permits assessment of diffusion components in specific directions.

As used herein, “force-based triggering” shall refer to acquisition of image data at pre-set force levels.

As used herein, “gating mechanism” shall refer to a device which reads the force transducer signal and produces trigger pulses for the scanner at pre-set force levels.

As used herein, “field strength” shall refer to the main magnetic field strength of the scanner.

The present invention provides a vaginal imaging probe (VIP) used as an internal and/or intravaginal probe for quantifying morphological and biochemical changes in the pelvic floor muscles and for monitoring muscular function. The vaginal imaging device (VIP) disclosed herein incorporates prior art design in terms of structural housing for the device. The initial vaginal probe was described by Dr. Arnold Kegel (6) in 1948 in the development of a perineometer for measuring pressure changes in response to pelvic floor muscle contraction. Although improvements in the perineometer have resulted in various shapes, sizes, and material composition, the overall concept of an insertable vaginal probe has been shown to be safe and efficacious.

The vaginal imaging probe of the present invention improves upon the previous design in structure to facilitate functional placement for maximal visualization and measurement of force conduction, and placement of a coil within the probe for imaging and spectroscopy. The vaginal imaging probe is the first to combine a resonator with a force measuring mechanism. This device can collect and analyze quantitative information regarding the mechanical function of the pelvic floor muscles that are responsible for attaining and maintaining urinary continence.

Pressure during contractions, magnetic resonance imaging (MRI), spectroscopy, and tagging of the levator ani musculature is monitored. This vaginal imaging probe acts as a dual frequency, that is proton and ³¹phosphorus, transmit/receive antenna for magnetic resonance (MR) imaging and spectroscopy of the musculature that contributes to urinary continence. Additionally, the vaginal imaging probe incorporates a force transducer to measure the force of muscular contractions and to permit triggering of image acquisitions according to the developed force levels via a force gating mechanism.

Such device allows analysis of biochemical changes, muscle density and morphology, and regional muscle function and strength in response to exercise of the pelvic floor musculature. More specifically, monitored biochemical changes include oxidative capacity, capillary bed blood flow, and mitochondrial content. Potential applications include, but are not limited to, description of the architectural morphology of the levator ani musculature, quantification of exercise-induced changes in the pelvic floor muscles such as muscle density, asymmetry during contractions and biochemical changes.

The vaginal imaging device includes variable capacitors with adjustment mechanisms and a dual tuned radiofrequency imaging/phosphorus spectroscopy coil. It is also contemplated that the vaginal imaging device may comprise a single tuned resonator single tuned resonator only for nuclear magnetic resonance imaging using the ¹H isotope. The housing for the device is likely to consist of an airtight, watertight hard plastic material that has a cylindrical shape and measures approximately 12.75 cm in length and 3 cm in diameter. Additionally, the imaging probe may be used rectally for diagnosis of fecal incontinence with an embodiment of this device in which the diameter of the disposable housing is reduced to 1.25-1.75 cm.

Two points on the housing consist of flexible plastic to permit contraction force transfer to the force transducer. A locator ring encircling the device is used for positioning purposes. The capability to measure contraction strength effort is integrated into the device using a piezoelectric force transducer, a resistive force transducer or an optical force transducer each with means to amplify the transduced signal. A cuff forms the base of the device to ensure correct placement and act as a leveling mechanism for freedom of probe movement during contractions.

The coaxial feed resonator consists of a dual resonant T-network matching circuit which permits impedance matching of the resonator to the characteristic impedance of the radiofrequency transmission network of the NMR scanner, i.e., 50 ohms. The tuning/matching circuit may be optionally located remote to the probe body. The transmit/receive element may be a single turn solenoid or an array of individual elements located around the long axis of the device. The vaginal imaging probe also has the ability to measure contractile force perpendicular to its long axis. Either antenna element is oriented such that the sensitive region corresponds to the muscles of interest, permitting the non-gradient localized spectroscopy to be performed.

This radiofrequency field profile also permits high resolution imaging to be performed through reduction of the field-of-view since the signal is inherently confined to the anatomy under consideration. Incorporated into the device is a piezoelectric, resistive or optical force transducer for measurement of developed contractile force. This provides monitoring of subject progress and a force proportional gating signal for the scanner which is necessary for imaging of mechanical function.

During operation, a radiofrequency signal fed to the vaginal imaging probe produces a corresponding radiofrequency magnetic field in the tissue of interest. The frequency of this field corresponds to the resonant frequency of the nuclei to be examined and produces a stimulated signal. This stimulated signal is received by the vaginal imaging probe which converts it to a corresponding electrical signal. This electrical signal is amplified and processed to produce image and spectroscopy data. Force transduction for measurement of muscular function is accomplished using a piezoelectric (charge proportional to force) or resistive bridge (voltage proportional to force) transducer or optical (light transmission proportional to force).

The device is designed for and has been demonstrated for use at 4.1 Telsa (T) (¹H frequency=174.86 MHz, ³¹P frequency=70.8 MHz); however, the device can also be used at 1.5 T (¹H frequency=63 MHz, ³¹P frequency=25.5. MHz) or at any field strength in clinical use. The most common main field strength in the base of installed clinical NMR scanners is 1.5 T. The primary sacrifice of the lower field strength is reduction of the signal to noise ratio for the phosphorus spectroscopy. However, the signal to noise ratio should remain adequate for diagnostic purposes as demonstrated in other applications, such as cardiac spectroscopy.

The vaginal imaging probe, by virtue of its ability to produce a confined radiofrequency (RF) magnetic field, permits imaging of the levator am muscles with a greater spatial resolution than is possible using conventional volume resonators. When the field-of-view on a MR scanner is set to be smaller than the object being imaged, signal from outside the defined region can “fold over” into the image field. In effect, when the field-of-view is set to be smaller than the object, the signals received are effectively under-sampled which manifests itself as “fold over”. The vaginal imaging probe, however, confines the RF excitation to a region within 2 centimeters of itself, permitting the field-of-view to be set to as little as. 4 centimeters, without concern for fold over, since tissue beyond this region is not being excited to produce any signal.

Spatial resolution improvement on the order of a factor of 6-10 over volume resonator imaging is therefore possible. The restricted transmit field allows setting of the field-of-view of the NMR scanner to its minimum value, thus maximizing spatial resolution without concern for fold-over artifact which can occur when NMR signals originate outside of the selected field-of-view. The element is designed to transmit and receive and is oriented to accomplish imaging and spectroscopy of the levator ani musculature through which the sphincter vaginae and compressor urethrae pass.

The prototype device was hand fabricated and assembled. The matching circuit and transmit/receive element were bench tested using a network analyzer to assess proper electrical function. Image and spectroscopy testing were carried out in phantoms to ensure proper localization of the radiofrequency magnetic field. Insulation properties were tested by raising input power to the dielectric breakdown limits of the capacitors in the matching circuit, i.e., 80 watts input power which is beyond the limits that would be applied in actual applications. The vaginal imaging probe is tested for current leakage according to normal hospital clinical biomedical engineering practice.

The vaginal imaging probe is designed for conducting intravaginal, non-invasive magnetic resonance imaging to measure strength, architectural morphology and biochemical analysis. The vaginal imaging probe also provides a mechanism for force activated triggering of the MR scanner to monitor regional muscle function and strength in response to exercise of the pelvic floor musculature. Potential applications include, but are not limited to, description of the architectural morphology of the levator ani musculature, quantification of exercise-induced changes in the pelvic floor muscles such as muscle density, asymmetry during contractions and biochemical changes.

Application of this device differs from current MRI techniques for examining the levator muscles by allowing 360 degrees of internal, intravaginal imaging and the ability to conduct biochemical analysis of the tissue, i.e., spectroscopy, along with muscle biomechanics information through high resolution RF tagged imaging techniques. More specifically, the biochemical changes monitored include oxidative capacity, capillary bed blood flow, and mitochondrial content. Additionally, radiofrequency (RF) tagged images are acquired at rest and at various contraction effort levels. The regional function of the muscles is reflected in the distortion of the tag lines. Thus, global depression of function, as well as regional abnormalities resulting from injury from, for example, childbirth or surgery, or from neurological dysfunction can be distinguished. Use of the vaginal imaging probe for functional muscle imaging requires use of the force triggering mechanism.

As provided herein, the vaginal imaging probe provides capabilities that exceed those currently available in urodynamic technology or standard magnetic resonance imaging (MRI). This device offers clinical investigators the instrumentation to examine several mechanisms of urinary control that have been beyond the state-of-the-science in continence research. Clinicians are able to individually quantitate the structural, metabolic, and biochemical dysfunction of each patient which has direct implications for the selection and development of the most cost-effective and appropriate treatment on a case-by-case basis.

The force-based triggering used in the probe also can be applied to ³¹P spectroscopy studies to examine the metabolic activity of the muscles during exercise at various levels of contraction effort. Investigation of the phosphorus metabolites and the pH provides a non-invasive, chemical “snap-shot” of the cellular metabolism. The design of the vaginal imaging probe allows non-gradient, localized phosphorus-31 spectroscopy of the levator ani muscles for assessment of metabolic function. The benefit of non-localized spectroscopy includes improved signal-to-noise ratio (SNR) and quantifiable improvement of temporal resolution over volume selective acquisitions.

Spectroscopy of these muscles enables acquisition of ³¹P MR spectra, which provides information in regard to intracellular levels of adenosine triphosphate (ATP), phosphocreatine (PCr), inorganic phosphate (Pi), phosphomonoesters, and phosphodiesters. Measurements of intracellular pH are also obtained from these ³¹p MR spectra. The vaginal imaging probe includes an internal phosphorus reference to permit absolute quantification of phosphorus metabolites.

Due to this non-invasive and painless technique, the need for muscle biopsy to describe the effects of exercise in biobehavioral treatment of urinary incontinence and determination of the mechanism of action for these non-surgical therapies is eliminated. The versatility of this design will enable the modification of the coil placement within the probe for visualization/imaging at or within the cervical os. This modification lends itself to diagnostic imaging and spectroscopy for studies of incompetent cervices in women who have repeated spontaneous abortions due to failure of the cervical os to retain the products of conception. In addition, use of the VIP in MR imaging and spectroscopy studies related to vaginal and cervical cancer and the effects of estrogen deficiency and supplemental estrogenation of the vaginal mucosa may provide knowledge regarding mechanism of action.

Previously, studies involving the circumvaginal musculature in regard to muscle fiber type and mechanism of action have been limited to in vivo biopsy or cadaver dissection. Imaging and spectroscopy using the vaginal imaging probe disclosed herein will also enable pre- and post-comparison of various techniques of surgical repair for bladder prolapse, cystocele repair, and pelvic sling procedures. Whereas surgical treatment is sometimes warranted, the efficacy of specific procedures remain in debate. This addition to the body of knowledge in treatment of urinary incontinence may provide quantifiable evidence of the efficacy for non-surgical treatments for incontinence, justification for Medical coverage and reimbursement to third party for these therapies.

The non-invasive nature of this technology lends itself to time-resolved investigations and repeat studies. Although muscle biopsies may provide similar information, these biopsies are often painful to subjects, are not conducive to studies that requires repeated measures and may result in additional destruction of tissue and nerves in an already compromised musculature. The present vaginal imaging probe is designed to solve the above problems.

As described herein, the invention provides a number of therapeutic advantages and uses. The embodiments and variations described in detail herein are to be interpreted by the appended claims and equivalents thereof. The following examples are given for the purpose of illustrating various embodiments of the invention and are not meant to limit the present invention in any fashion.

### EXAMPLE 1

Vaginal Imaging Probe Design with Pneumatic Force Transduction

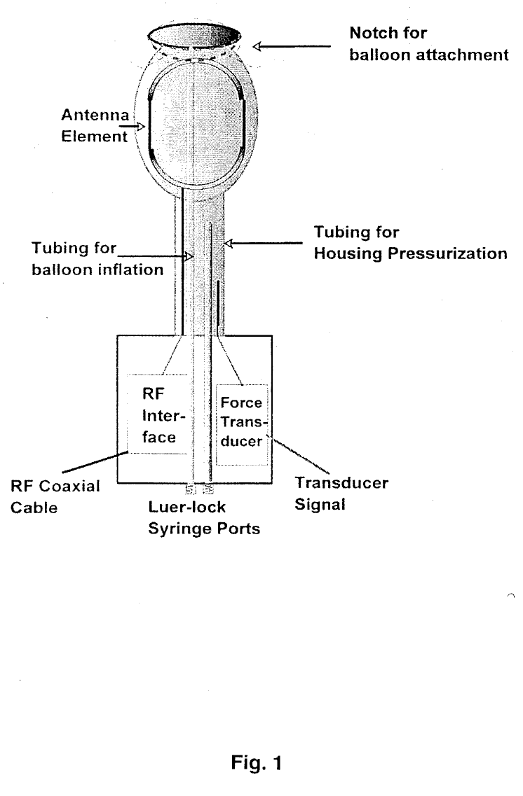

FIG. 1 shows the components for a vaginal imaging probe housing containing a single turn solenoid antenna and a pneumatic pressure transducer. The probe comprises a permanent, non-disposable housing unit enclosing the RF tuning/matching circuit or radiofrequency interface with coaxial cable, the pressure transducer and interconnections for the same. The probe further comprises a disposable component containing the antenna element and, optionally, a small glass vial containing a solution of inorganic phosphate mounted at the center of the loop (not shown). The attachment of the disposable portion of the probe to the main housing is made through an airtight fitting to permit the internal volume of the disposable housing to function as a changeable volume for pressure transduction.

With regard to the pneumatic pressure transducer for contraction force measurement a representative device is the Motorola MPX10 series of silicon film pressure transducers. A small tube within the housing connects the pressure port of the transducer to a port on the housing where communication is made with the volume enclosed by the disposable housing containing the antenna element. Provision is made for delivery of a DC bias signal to the transducer, e.g., for the Motorola MPX series, a 5V bias can be used, and for obtaining the force proportional output signal for delivery to a gating unit to synchronize the MRI scanner. Also contained in the main housing is a tube allowing connection of a syringe to add air pressure to the disposable housing to provide for mechanical stabilization once inserted.

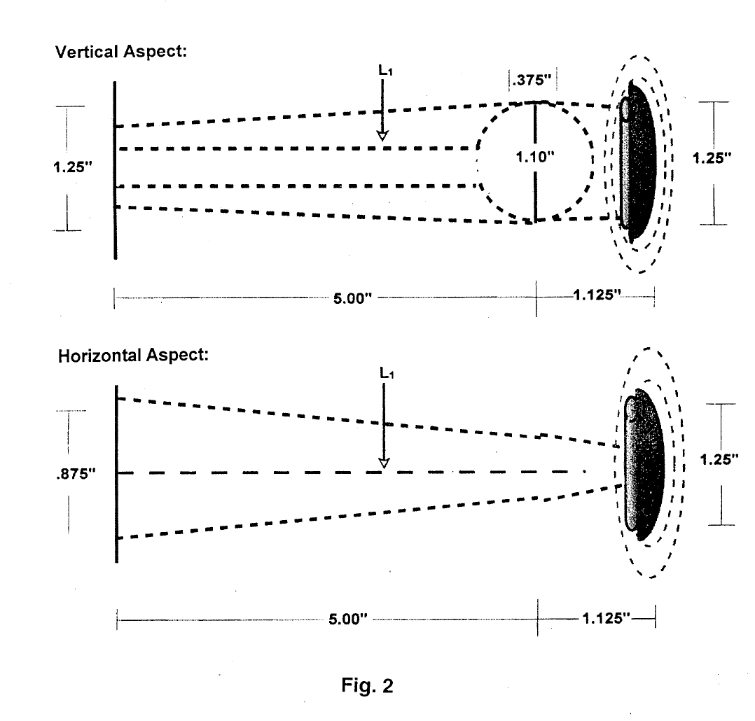

FIG. 2 depicts the layout and geometry of the disposable component of the probe. The disposable component has a compliant, hollow housing. Contraction force exerted by the subject results in a change in the volume of the compliant housing, increasing its internal air pressure. This change in pressure is detected by the force transducer and reflected in its output signal. Pressure measurements can be made continuously during the course of a study. The disposable housing also incorporates an annular inflatable ring at its end which expands under pressurization to provide for mechanical stabilization

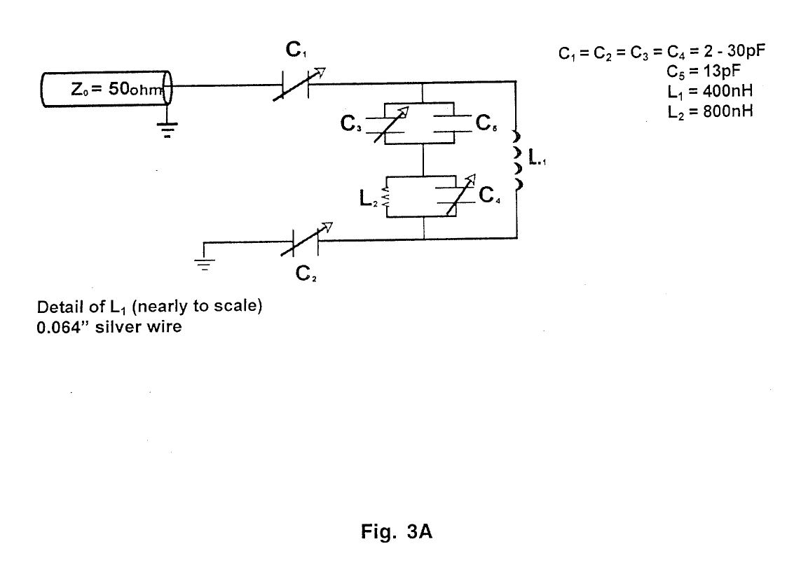

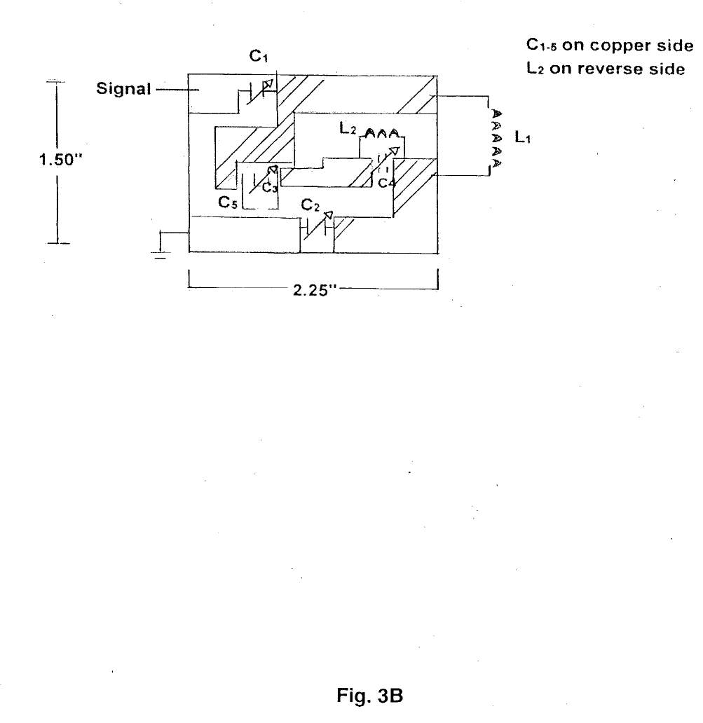

FIG. 3A depicts the radiofrequency tuning/matching circuit. the tuning/matching circuit is intended to produce a 50Ω input impedance at two different resonant frequencies, corresponding to the ¹H and, typically, ³¹P resonant frequencies. The second nucleus can also be selected to be ²³Na, ¹³C, etc. for purposes of spectroscopic studies. The two variable capacitors in series with the two coaxial cable conductors (C₁ and C₂) are primarily responsible for establishing the input impedance, whereas the remaining capacitors (C₃ and C₄) are primarily responsible for establishing the frequencies of the resonances ( defined as the frequencies where an input impedance of 50Ω is achieved). FIG. 3B depicts the circuit mounted on a printed circuit board.

With continued reference to FIGS. 3A-3B it is contemplated that, in an alternate embodiment of this device, the tuning/matching circuit is single tuned. In this embodiment, typically, ¹H is used for nuclear magnetic resonance imaging purposes. In this embodiment, the parallel capacitor/inductor combination,L₂-C₄, is deleted.

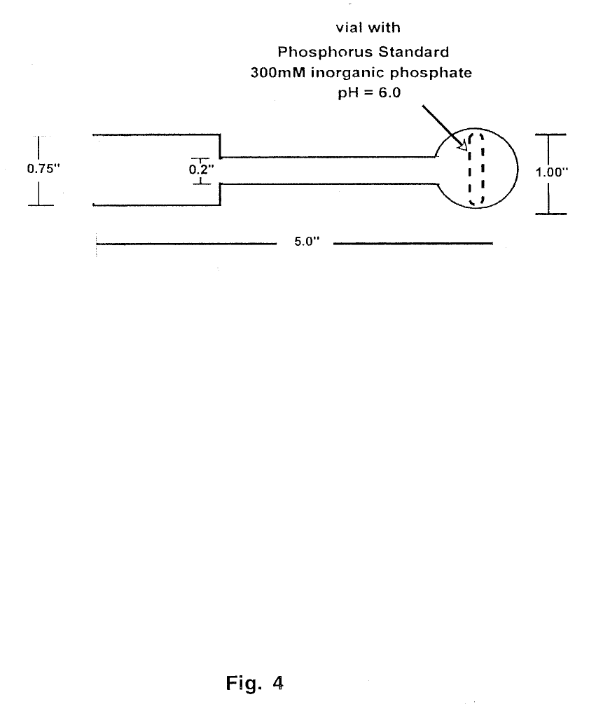

FIG. 4 depicts a single turn solenoid antenna element. Mounted at the center of the loop is a small glass vial containing a 300 mM solution of inorganic phosphate at pH 7.4. This solution acts as a chemical shift reference for phosphorus spectroscopy to permit positive identification of the ATP and PCr peaks in the muscle spectra and to provide a reference for deriving tissue pH from the chemical shift difference between PCr and inorganic phosphate which has a pH dependence. Alternatively, the antenna element consists of multiple loops established as a phased array to alter the sensitivity profile of the device for more uniform coverage around the long axis of the probe.

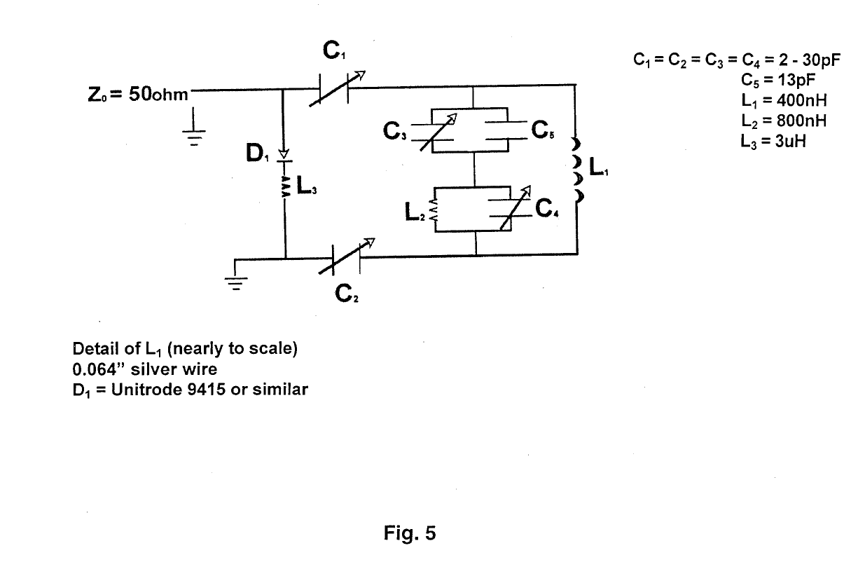

FIG. 5 depicts the probe as a receive-only resonator. Active and passive decoupling circuits can be included in the circuitry. In the active mode, the PIN diode is brought into conduction by a bias signal sent by the scanner. This causes bypass of C₃ shifting the resonant frequency upward, and making the probe non-responsive at the imaging frequency, thereby preventing local retransmit of the radiofrequency excitation originating from the scanner's volume resonator.

As a redundancy mechanism, a passive decoupling circuit consists of a pair of crossed PIN diodes across L₁. This mechanism does not require a bias signal from the scanner. When the volume resonator transmits, the diodes go into conduction and bypass L₁ resulting in a change in the resonant frequency, making the probe non-responsive at the imaging frequency and preventing local retransmit of the RF excitation originating from the scanner's volume resonator again, to prevent undesired tissue heating.

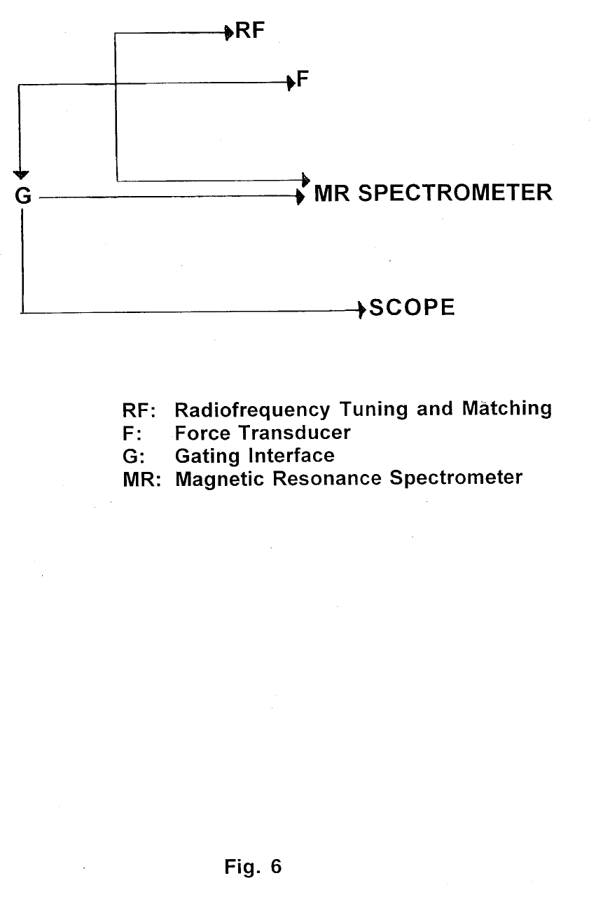

FIG. 6 depicts the interconnections and relationships between the components of the pneumatic force transduction probe and the MRI scanner. A gating interface is used to provide DC bias to the pressure transducer in the main housing, and to receive and process the force proportional signal delivered by the transducer. The gating interface, which is connected to the scanner, can be used to issue control signals to the scanner when the patient exerts specified levels of contraction effort. A 50Ω coaxial cable delivers the RF transmit excitation to the probe and carries the received NMR signal to the scanner receiver. When the probe is used as a receive-only device, the coaxial cable only delivers the received NMR signal to the scanner receiver.

### EXAMPLE 2

Helmholz Array Probe with Fiber Optic Force Transduction Mechanism and Remote Tuning Arrangement

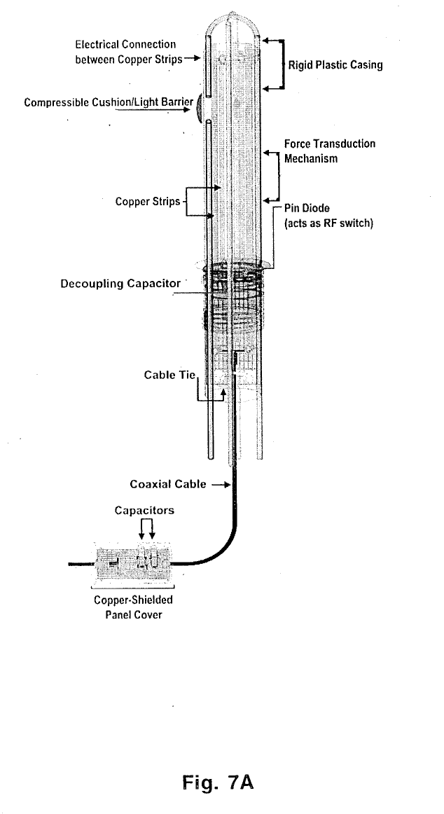

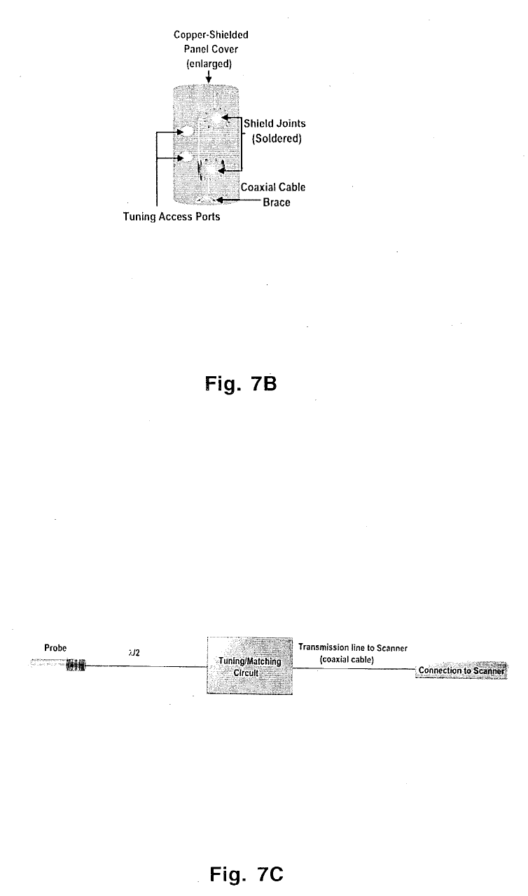

FIG. 7 depicts the components for a vaginal imaging probe containing a Helmholz array antenna and an optical force transducer. Generally, the probe comprises a permanent, nondisposable housing unit and a disposable component. The probe is connected to a remote tuning box that contains the tuning/matching network. The tuning/matching circuit used in the network is the same as depicted in FIG. 3A and can be either dual tuned or single tuned as previously described.

The disposable component of the probe has a primarily rigid plastic casing. The disposable component contains fiber optic paths recessed therein and deformable pads for force transduction located across gaps in the optical fiber filaments recessed within the probe surface. The helmholz antenna is beneath the transduction layer and within the plastic casing. It connects to the external housing with contacts for the antenna elements and fiber optics and locks in place. Alternatively the disposable part of the probe consists only of the optical transduction components which slide over a rigid form. The antenna elements are on its surface in which case only optical connections are made when the disposable part is installed.

The antenna elements comprise a Helmholz array. Individual copper strips are positioned around the long axis of the probe and are electrically connected. The Helmholz array provides a different spatial sensitivity profile when compared to a single turn solenoid antenna.

The fiber optic filaments function as an optical force transduction mechanism. Since the signals are carried by optical fibers, no electrical interaction between the probe antenna and the force transduction mechanism occurs during operation. During force transduction, force exerted by a patient acts on the deformable pads which progressively occlude the light path across the gap in the optical fiber path as force develops. Light transmission in this instance is therefore inversely proportional to force. It is contemplated that optical force transduction can be used in a manner to either positively or negatively correlate to applied force.

Two transduction points are placed 90 degress apart on the probe to permit detection of anterior-posterior or left-right force development. Light emitting diodes are used as the sources and phototransistors act as the detectors (not shown). Once in the form o felectrical signals, normal triggering criteria can be set to select either the anterior-posterior or left-right channel or to trigger if either channel passes the selected force threshold.

The non-disposable housing unit encloses a decoupling capacitor which functions when the probe or resonator is single tuned, as with the previously described for a probe comprising a single turn solenoid, a pin diode that acts as an RF switch, the coaxial cable which connects to the remote tuning box, and a cable tie for the coaxial cable. The tuning/matching circuit in the network in the remote tuning box is connected to the probe body by a coaxial transmission line of electrical length λ/2 wavelength or and integer multiple thereof. The remote tuning box comprises a copper-shielded panel cover.

FIG. 7B depicts an enlargement of the panel cover which comprises soldered shield joints, tuning access ports and a coaxial cable brace.

FIG. 7C depicts the remote tuning layout. The layout shows the remote tuning box connected to the probe by a λ/2 wavelength electrical length of coaxial transmission line. Coaxial cable is also used to connect the remote tuning box to the scanner. Thus the tuning/matching circuit is located away from the probe body thereby facilitating patient comfort and ease of tuning by a technician. This does not preclude a remote tuning/matching circuit compatible with automatic tuning features offered by specific scanners.

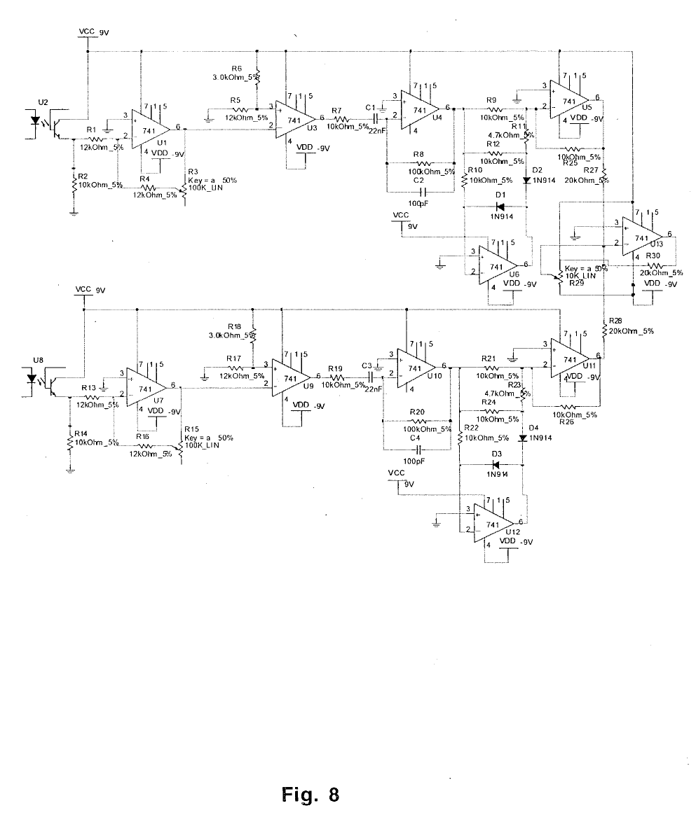

FIG. 8 depicts the circuit diagram for the optical force transduction mechanism used in the Helmholz array. In this arrangement the gate signal is taken from pin 6 of U12.

The following references were cited herein.

1. Astrand, P O & Rodahl, K (1986). Textbook of work physiology: Physiological bases of exercise. New York: McGraw-Hill Book Company.

2. Hortobagyi, T et al. (1991). The Journal of Sports Medicine and Physical Fitness, 31(1), 20-30.

3. Critchley, H O D et al. (1980). Urologia Internationalis, 35, 226-232.

4. Gilpin, S A et al. (1989). British Journal of Obstetrics and Gynecology, 96, 15-23.

5. Parks, A G et al. (1977). Gut, 18, 656-665.

6. Kegel, A H (1948). American Journal of Obstetrics and Gynecology, 56, 238-248.

7. Jones, E G & Kegel, A H (1952). Surgery, Gynecology, and Obstetrics, 94, 179-188.

8. Kegel, A H (1951). Journal of the American Medical Association, 146, 915.

9. Kegel, A H (1956). Journal International College of Surgeons, 25, 487.

10. Kegel, A H & Powell, T O (1950). Journal of Urology, 63, 808-814.

11. Bo, K et al (1990). Neurourology and Urodynamics, 9, 489-502.

12. Burns, P A et al. (1993). Journal of Gerontology: Medical Sciences, 48(4), M167-M174.

13. Dougherty, M et al. (1993). Journal of Reproductive Medicine, 38(9), 684-691.

14. Wells, T (1990). Journal of the American Geriatric Society, 38(3), 333-337.

15. Johnson V Y (2001). Effects of a submaximal exercise protocol to recondition the pelvic floor muscles, 50(1), 33-41.

16. Christensen, L L et al. (1995). Neurourol Urodyn, 14(3), 209-216.

17. Bartolozzi, C et al., (1996). Eur. Radiol., 6(3): 339-345.

18. Bates, T S et al., (1996). Clin. Radiol., 51(8): 550-553.

19. Boska, M (1991). NMR in Biomed., 4: 173-181.

20. Boska, M. (1994). Magn. Reson. Med., 32: 1-10.

21. Gufler, H et al. (1999). J. Magn. Reson. Imaging, 9(3), 378-83.

22. Huch Boni, R A et al. (1995). J. Compu. Assist. Tomogr., 19(2): 232-237.

23. Kurhanewicz, J et al. (1991). Magn. Reson. Med., 22(2): 404-413.

24. Larson, D E et al. (1997). The effect of weight reduction on ATP production rate and metabolic economy during 90-second isometric plantar flexion exercise at 50% of theoretical maximal voluntary contraction force output. “Proc., ISMR, 5^{th} Annual Meeting, 1997” p. 1302.

25. Newcomer, B et al. (1997). Muscle and Nerve, 20: 336-346.

26. Vanbeckevoort, D (1999). J. Magn. Reson. Imaging, 9(3), 373-377.

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A vaginal imaging device, comprising:
a single tuned or dual tuned resonator comprising a transmit/receive element for nuclear magnetic resonance imaging or nuclear magnetic resonance and spectroscopy;
a tuning/matching network remote from said resonator; and
a force transduction mechanism for monitoring a subject's contraction effort and to trigger said resonator to produce vaginal imaging and spectroscopy data.

2. The vaginal imaging device of claim 1, wherein said tuning/matching circuit is electrically connected to said resonator via a coaxial transmission line having an electrical length of λ/2 wavelength or an integer multiple thereof.

3. The vaginal imaging device of claim 1, wherein said single tuned resonator is for nuclear magnetic resonance imaging using the ¹H isotope.

4. The vaginal imaging device of claim 1, wherein said dual tuned resonator is for ¹H isotope imaging and for performing nuclear magnetic resonance spectroscopy of a second isotope selected from the group consisting of ³¹P, ¹³C, ²³Na, ³⁹K, and ⁴³Ca.

5. The vaginal imaging device of claim 1, wherein a vial containing a 300 mM inorganic phosphate reference solution is located at the center of said transmit/receive element in said resonator to allow chemical shift referencing for the signals obtained.

6. The vaginal imaging device of claim 1, wherein said transmit/receive element is a single turn solenoid oriented to permit non-gradient localized spectroscopy.

7. The vaginal imaging device of claim 1, wherein said transmit/receive element is a Helmholz array located around the long axis of the device for the purpose of providing a different spatial sensitivity profile than that provided by a single turn solenoid.

8. The vaginal imaging device of claim 1, wherein said force transduction mechanism is an optical force transducer, a piezoelectric force transducer, a resistive force transducer or a pneumatic pressure transducer.

9. The vaginal imaging device of claim 1, wherein said force transduction mechanism is used to monitor contraction effort of the subject for the purpose of synchronizing scanner image or spectroscopy data acquisition with the contraction effort of the subject.

10. The vaginal imaging device of claim 9, wherein said force transduction mechanism is used to synchronize scanner image acqusition with the scanner body volume resonator.

11. The vaginal imaging device of claim 9, wherein said force transduction mechanism is used to synchronize scanner image or spectroscopy data acquisition with its own antenna element in a transmit/receive mode.

12. The vaginal imaging device of claim 9, wherein said force transduction mechanism is used to synchronize scanner image or spectroscopy data acquisition with its own antenna element in a receive only mode with active or passive decoupling, wherein said decoupling prevents local retransmission of the radiofrequency signal and excessive tissue heating.

13. The vaginal imaging device of claim 1, wherein said imaging is radiofrequency tagged magnetic resonance imaging, phase velocity mapping or diffusion weighted imaging.

14. The vaginal imaging device of claim 1, wherein said spectroscopy is non-gradient localized phosphorus spectroscopy.

15. A vaginal imaging device, comprising:
a single tuned or dual tuned resonator for nuclear magnetic resonance imaging or nuclear magnetic resonance and spectroscopy comprising a Helmholz array;
a tuning/matching network remote from said resonator; and
an optical force transducer that generates a gating signal from an optical signal proportional to developed force, said gating signal triggering said resonator to produce vaginal imaging and spectroscopy data.

16. The vaginal imaging device of claim 15, wherein said tuning/matching network is electrically connected to said resonator via a coaxial transmission line having an electrical length of λ/2 wavelength or an integer multiple thereof.

17. The vaginal imaging device of claim 15, wherein said single tuned resonator is for nuclear magnetic resonance imaging using the ¹H isotope.

18. The vaginal imaging device of claim 15, wherein said dual tuned resonator is for ¹H isotope imaging and for performing nuclear magnetic resonance spectroscopy of a second isotope selected from the group consisting of ³¹P, ¹³C, ²³Na, ³⁹K, and ⁴³Ca.

19. The vaginal imaging device of claim 15, wherein a vial containing a 300 mM inorganic phosphate reference solution is located at the center of said Helmholz array in said resonator to allow chemical shift referencing for the signals obtained.

20. The vaginal imaging device of claim 15, wherein said imaging is radiofrequency tagged magnetic resonance imaging.

21. The vaginal imaging device of claim 15, wherein said spectroscopy is phosphorus spectroscopy.

22. A method for imaging pelvic floor musculature in a subject, comprising the step of:
applying the vaginal imaging device of claim 1 to said subject to produce an image of the pelvic floor musculature.

23. A method for imaging pelvic floor musculature in a subject, comprising the step of:
applying the vaginal imaging device of claim 15 to said subject to produce an image of the pelvic floor musculature.

24. A method for obtaining spectroscopic information on the biochemical state of pelvic floor musculature in a subject, comprising the step of:
applying the vaginal imaging device of claims 1 to said subject to produce magnetic resonance spectroscopic information which provides assessment of muscular biochemical activity.

25. A method for obtaining spectroscopic information on the biochemical state of pelvic floor musculature in a subject, comprising the step of:
applying the vaginal imaging device of claims 15 to said subject to produce magnetic resonance spectroscopic information which provides assessment of muscular biochemical activity.

26. A method for assessing biochemical changes under exercise conditions in pelvic floor musculature in a subject, comprising the steps of:
a) applying the vaginal imaging device of claims 1 to said subject at rest to acquire magnetic resonance spectroscopic data;
b) applying said vaginal imaging device to said subject during exercise to acquire magnetic resonance spectroscopic data;
c) applying said vaginal imaging device to said subject after exercise to acquire magnetic resonance spectroscopic data; and
d) comparing the data collected in (a), (b) and (c), wherein said comparison provides assessment of biochemical changes under exercise conditions in pelvic floor musculature in said subject.

27. A method for assessing biochemical changes under exercise conditions in pelvic floor musculature in a subject, comprising the steps of:
a) applying the vaginal imaging device of claims 15 to said subject at rest to acquire magnetic resonance spectroscopic data;
b) applying said vaginal imaging device to said subject during exercise to acquire magnetic resonance spectroscopic data;
c) applying said vaginal imaging device to said subject after exercise to acquire magnetic resonance spectroscopic data; and
d) comparing the data collected in (a), (b) and (c), wherein said comparison provides assessment of biochemical changes under exercise conditions in pelvic floor musculature in said subject.

28. A method for evaluating efficacy of a surgical repair in pelvic floor musculature in an individual, comprising the steps of:
applying the vaginal imaging device of claim 1 to said individual before the surgical repair to produce a pre-surgery image of the pelvic floor. musculature;
applying said vaginal imaging device to said individual after the surgical repair to produce a post-surgery image of the pelvic floor musculature; and
comparing said post-surgery image with said pre-surgery image, wherein differences in said images are indicative of the efficacy of said surgical repair.

29. A method for evaluating efficacy of a surgical repair in pelvic floor musculature in an individual, comprising the steps of:
applying the vaginal imaging device of claim 15 to said individual before the surgical repair to produce a pre-surgery image of the pelvic floor musculature;
applying said vaginal imaging device to said individual after the surgical repair to produce a post-surgery image of the pelvic floor musculature; and
comparing said post-surgery image with said pre-surgery image, wherein differences in said images are indicative of the efficacy of said surgical repair.

30. A method for evaluating efficacy of an exercise therapy in an individual, comprising the steps of:
applying the vaginal imaging device of claim 1 to said individual before said exercise therapy to produce a pre-therapy image of the pelvic floor musculature;
applying said vaginal imaging device to said individual after said exercise therapy to produce a post-therapy image of the pelvic floor musculature; and
comparing said post-therapy image with said pre-therapy image, wherein differences in said images are indicative of the efficacy of said exercise therapy.

31. A method for evaluating efficacy of an exercise therapy in an individual, comprising the steps of:
applying the vaginal imaging device of claim 15 to said individual before said exercise therapy to produce a pre-therapy image of the pelvic floor musculature;
applying said vaginal imaging device to said individual after said exercise therapy to produce a post-therapy image of the pelvic floor musculature; and
comparing said post-therapy image with said pre-therapy image, wherein differences in said images are indicative of the efficacy of said exercise therapy.

32. A method for evaluating efficacy of a pharmaceutical therapy in an individual suffering from abnormalities in pelvic floor musculature, comprising the steps of:
applying the vaginal imaging device of claim 1 to said individual before said pharmaceutical therapy to produce pre-therapy magnetic resonance spectroscopic data;
applying said vaginal imaging device to said individual after said pharmaceutical therapy to produce a post-therapy magnetic resonance spectroscopic data; and
comparing said post-therapy data with said pre-therapy data, wherein differences in said images are indicative of the efficacy of said pharmaceutical therapy.

33. A method for evaluating efficacy of a pharmaceutical therapy in an individual suffering from abnormalities in pelvic floor musculature, comprising the steps of:
applying the vaginal imaging device of claim 15 to said individual before said pharmaceutical therapy to produce pre-therapy magnetic resonance spectroscopic data;
applying said vaginal imaging device to said individual after said pharmaceutical therapy to produce a post-therapy magnetic resonance spectroscopic data; and
comparing said post-therapy data with said pre-therapy data, wherein differences in said images are indicative of the efficacy of said pharmaceutical therapy.

34. The vaginal imaging device of claim 15, wherein said device is suitable for rectal use in cases of fecal incontinence.
